(19) United States
(12) Patent Application Publication
(10) Pub. No.: US 2009/0023662 A1
(43) Pub. Date: Jan. 22, 2009

(54) Identification of Agonistic Autoantibodies Associated with Humoral Kidney Rejection

(75) Inventor: Gerd Wallukat, Berlin (DE)

Correspondence Address: NORRIS, MCLAUGHLIN & MARCUS, P.A., 875 THIRD AVE, 18TH FLOOR, NEW YORK, NY, 10022, US

(73) Assignee: MAX-DELBRUECK-CENTRUM FUER MOLEKULARE MEDIZIN, Berlin (DE)

(21) Appl. No.: 11/948,064

(22) Filed: Nov. 30, 2007

Related U.S. Application Data
(62) Division of application No. 10/536,552, filed on Jan. 26, 2006.

(30) Foreign Application Priority Data
Nov. 29, 2002 (DE) 102 56 897.9
Jan. 27, 2003 (DE) 103 03 120.0
Jun. 13, 2003 (DE) 103 27 066.3

(51) Int. Cl. G01N 33/53; A61K 38/08; A61P 37/00; A61P 41/00

(52) U.S. Cl. 514/16; 435/7.1; 514/17; 436/501

(57) ABSTRACT

The invention relates to a method for detecting autoantibodies associated with humoral kidney rejection, which recognize extracellular structures of G protein-coupled receptors, and to the use of peptides, which comprise these loops or fragments thereof, for treating humoral kidney rejection.

The invention relates to a method for detecting autoantibodies associated with humoral kidney rejection, which bind loops of G protein-coupled receptors, and to the use of peptides, which comprise these loops or fragments thereof, for treating humoral kidney rejection.

The immune system of multicellular organisms is based on the differentiation between “self” and “not self”. Because of its great diversity, the immune system that differentiates between “self” and “not self” has at its disposal a large repertoire of specificities, which are expressed particularly by T cells and B cells. By means of complicated mechanisms, the immune system is enabled to differentiate between “self” and “not self,” with which mechanisms the body can protect itself, in particular, against the consequences of so-called autoimmunity. This means that the humoral and cellular components of the immune system of an organ are marked in such a manner that they do not direct themselves against the organism itself. However, many different types of disturbances can occur within the immune system; in particular, the mechanisms of self-recognition can be restricted or completely eliminated. Therefore there are a number of diseases that can be triggered by autoantibodies or autoreactive T cells. One of the first diseases in which autoantibodies against a specific organ were found, is Hashimoto thyroiditis. This is a disease of the thyroid that occurs mainly in middle-aged women and leads to the formation of a goiter and hypofunction of the thyroid. If the disease is not treated, complete destruction and shrinkage of the organ occur. Aside from autoantibodies that are directed exclusively against an organ, there are numerous autoantibodies that can be directed against several organs or tissue types.

In accordance with the orientation of the autoantibody, a differentiation can be made between organ-specific and non-organ-specific autoimmune diseases. Typical organ-specific autoimmune diseases are, for example, premature menopause, juvenile diabetes, male infertility, pernicious anemia or Addison's disease. The non-organic specific autoimmune diseases include, for example, rheumatoid arthritis, dermatomyositis, scleroderma, or mixed connective tissue diseases and others. Frequently, target organs affected by organ-specific disease are the thyroid, the adrenal gland, the stomach, and the pancreas, while the non-organ-specific diseases are summarized by the term of the so-called rheumatic form group, and relate to the skin, the kidneys, the joints and the muscles. Up to the present, only a few, inadequate methods have become known for the diagnosis and treatment of autoimmune diseases. Using the known laboratory routines, such as ELISA, or other diagnostic methods that have become established, which have proven themselves in mass screening under the laboratory conditions of a clinic, for example, it is not possible to detect the autoantibodies in the serum of a patient, which are often present only in small concentrations. Treatment in the case of organ-specific diseases takes place, in most cases, by restoring the metabolic equilibrium; for example, in the case of thyroid hypofunction, the missing thyroid hormone is substituted with thyroxin, and in the case of thyrotoxicosis, antimetabolites of the hormone can be administered. In pernicious anemia, a depot of vitamin B₁₂ can be administered parenterally, and in myasthenia gravis, cholinesterase inhibitors can be administered. If a complete loss of function of an organ has occurred, an organ transplant or implantation of a prosthesis, for example, are possible. Such therapeutic methods are not suitable in the case of non-organ-specific autoimmune diseases, since here, for example, an entire group of organs, such as the skin, the kidneys, the joints, and the muscles, would have to be substituted in a patient, whereby substitution of the joints, the muscles and/or the skin, alone, is almost impossible. Another possibility for treating autoimmune diseases is to bind the autoantibody that induces the disease, to complex it, and consequently to eliminate it from the serum. Such methods can, however, only be successfully used in diagnosis or therapy if a target, an agent, or a structure are known, with which the autoantibodies interact in such a manner, that they can be detected or eliminated using the agent or the target. For numerous autoimmune diseases, for example humoral kidney rejection, such agents are not known. Previous diagnosis methods, such as bioassays, are difficult to handle and are therefore not suited for laboratory routine. Known bioassays are, for example, cardiomyocyte cultures for detecting angiotensin II AT1 receptor autoantibodies.

It was therefore an object of the invention to make available means, devices, and methods for the diagnosis of autoantibodies associated with humoral kidney rejection and the treatment of humoral kidney rejection, which allow simple, efficient, and reliable detection or treatment, and which do not demonstrate the disadvantages stated.

The invention solves this technical problem by means of making available a method for the detection of autoantibodies associated with humoral kidney rejection, which are directed against G protein-coupled receptors, whereby the method comprises the following steps:
a) Bringing bodily fluid into contact with a denaturing agent,
b) Bringing the precipitated fraction into contact with a peptide, particularly one comprising biotin, which comprises a partial sequence of the first and/or second loop of the receptor, whereby a mixture is formed,
c) Incubating the mixture with a carrier, particularly one coated with avidin or streptavidin,
d) Washing the materials of the carrier,
e) Incubating the carrier with anti-IgG subclasses, whereby the anti-IgG antibody is marked, and
f) Carrying out a detection reaction, particularly an enzyme reaction or color reaction.

In other words, the invention relates to the surprising teaching that it is possible to detect autoantibodies associated with humoral kidney rejection disease, which are particularly directed against G protein-coupled receptors, using an enzyme reaction or color reaction, for example in an ELISA, a customary laboratory routines. Preferably, the antibodies are agonistic autoantibodies. Preferably, this disease can be detected simply, more reliably and effectively using an inventive enzyme-coupled immune test. This disease of humoral kidney rejection, particularly the antibodies associated with it, could only be detected, until now, using complicated indirect tests, such as bioassays. Thus, angiotensin II-AT1 receptor autoantibodies, which are associated with preeclampsia and humoral kidney rejection, for example, were detected reliably until now only by bringing the sera, in each instance, into contact with cultivated cardiomyocytes, particularly those of newborn rats, by way of a modification in the heart rate. For this purpose, it was necessary to culture the cardiomyocytes of newborn rats and to bring them into contact with the serum of the patients, whereby antibodies against the AT1 receptor could be detected in patients having preeclampsia, by means of the detection of the increase in the beats per minute. Using the inventive method, an immune test is made available with which autoantibodies associated with humoral kidney rejection that are directed against G protein-coupled receptors can be detected. These autoantibodies are preferably an angiotensin II AT1 receptor autoantibody. Such a streptavidin-coated carrier is a carrier, which is coated preferably with streptavidin or avidin. Particularly preferred are proteins consisting of 6 to 2, particularly 4 subunits, M_{R} approximately 40,000 to 80,000, particularly 60,000 or 66,000, isoelectric point close to the neutral point, such as streptavidin or avidin, for example, which demonstrate a high affinity (e.g. K_{D}=10⁻¹⁵ M⁻¹) to other compounds, particularly those inactivating by means of choline, preferably biotin. It is known to a person skilled in the art that a carrier, particularly one coated with streptavidin, is advantageous, if a structure that can be connected in effect with the latter is associated with biotin or an equivalent. If it is determined experimentally that the bond is sufficient for detection even without a biotin/streptavidin/avidin association or bond, the peptide or the carrier does not have to be bound to these ancillary substances (biotin and streptavidin/avidin).

In the sense of the invention, the peptide is a molecule, which consists essentially of amino acids. Peptides in the sense of the invention are also structures, which comprise more than 50 or 100 amino acids, respectively, and therefore can also be referred to as proteins. Peptides and proteins are therefore used synonymously in connection with the invention. Of course, the peptides may comprise other structures such as lipids or carbohydrates, but also artificial or natural amino acid modules or non-amino acid modules.

The peptide or protein comprising biotin can also be combined with a tag other than biotin. In the sense of the invention, a tag is a protein tag or a peptide or another structure, which is combined with, for example, fused to the peptide or protein. The peptide may, as already explained, be present in biotinylated form, and therefore have biotin as the tag. A person skilled in the art is familiar with other tag structures or tags, from catalogs and standard works in biochemistry. Preferred tags are His tag, Flag tag, Strep tag, T7 tag (1 N-terminal amino acids of the T7Gen10 protein), S tag, CBP (calmodulin binding peptide), MBP (maltose binding peptide), Neb, Protein A, GST tag, PinPoint tag, thioredoxin, PET (cellulose binding domain), Pmal (maltose binding domain), and/or biotin tag. This tag possesses a high affinity for an anti-tag substance or an anti-tag on the carrier. Preferred anti-tags are streptavidin, glutathione, biotin, nickel-NTA, cellulose, amylose, thiobond, avidin, and/or immunoglobulin. A person skilled in the art is familiar with tag/anti-tag pairs, i.e. the selection of the tag determines the structure of the anti-tag, without an inventive selection by the person skilled in the art being required, or such person first having to solve a technical task in the implementation of the inventive teaching. If the peptide is present in biotinylated form, and therefore demonstrates biotin as the tag, the anti-tag structure is streptavidin, i.e. a carrier coated with streptavidin. Other preferred tag/anti-tag pairs are: Protein A/immunoglobulin, GST (glutathione S transferase)/glutathione, Pin-Point (in vivo biotinylation)/avidin, thiorexidin/thiobond, PET (cellulose binding domain)/cellulose, and/or Pmal (maltose binding domain)/amylose, and others. Preferred carriers coated with anti-tag are selected from the group comprising carriers coated with streptavidin, glutathione, biotin, nickel-NTA, cellulose, amylose, thiobond, avidin, and/or immunoglobulin.

The inventive loops may be extracellular structures with which functional, agonistic, or antagonistic autoantibodies interact, detecting or binding them.

In a particular embodiment of the invention, the denaturing agent is ammonium sulfate. Of course, however, any denaturing agent that does not change the structure much can be used, such as alcohol in alcohol precipitation, for example. Advantageously, using ammonium sulfate or alcohol, it is possible to precipitate bodily fluids, such as serum, for example, particularly to precipitate them in fractionated manner. Thus, for example, autoantibodies can be separated from other components of the bodily fluid. Denaturing of the bodily fluid takes place, in this connection, particularly in such a manner that the separated components can be returned to essentially the native state, or to a state that allows their detection, by means of methods known to a person skilled in the art. Of course, any denaturing agent known to a person skilled in the art, which is different from ammonium sulfate, or another denaturing agent that does not change the structure much is suitable for precipitating bodily fluids.

In another preferred embodiment of the invention, the carrier is a magnetic particle or an ELISA plate or another structure, which is suitable for incubating the mixture. Magnetic particles, in particular, permit the bound mixture to be separated using current or a magnetic charge. The use of ELISA plates preferably permits the use of laboratory routines and standardized equipment, since ELISA plates, particularly 96-well microtiter plates, are used as a standard in clinical and basic research.

In the present invention, the autoantibody is directed against an angiotensin II AT1 receptor, and in particular, which receptor is associated with, humoral kidney rejection. The autoimmune disease of humoral kidney rejection is one in which the disease-associated autoantibodies are directed against certain extracellular structures of the G protein-coupled receptor. This disease been difficult or impossible to diagnose using previous means of laboratory routine, and, furthermore, can be treated with only significant effort, in some cases surgical effort (e.g. heart transplants or implantation of heart support systems), particularly, owing to the fact that individual tissue, organ regions, or complete organs are substituted by prostheses or other organs, for example, from living or dead patients.

In a very special embodiment of the invention, the peptide that comprises a sequence or partial sequence of the second loop of the receptor is preferably used for humoral kidney rejection. It is advantageously possible to use the first and/or second loop, or only the second loop, or peptides that comprise portions or fragments of the first and/or second loop, or only of the second loop, for detecting or treating the stated disease. Advantageously, a person skilled in the art is given various possibilities for diagnosing, prognosticating, treating the stated autoimmune disease, for follow-up treatment, or monitoring the progression of the treatment method, in each instance, during the course of treatment, by means of the disclosure of the connection between autoantibody, loop and the autoimmune disease, in each instance. The loops, i.e. the peptides that comprise partial regions of the loops, preferably are modified.

It is known to a person skilled in the art that individual amino acids demonstrate analogous physicochemical properties, which advantageously lead to the result that these amino acids can be substituted for one another. These include, for example, the group of amino acids (a) glycine, alanine, valine, leucine, and/or isoleucine; or the amino acids (b) serine and threonine; the amino acids (c) asparagines and glutamine; the amino acids (d) asparaginic acid and glutaminic acid; the amino acids (e) lysine and arginine; as well as the group of aromatic amino acids (f) phenyl alanine, tyrosine, and/or tryptophan. Amino acids within one and the same group (a-f) can be replaced for one another. Furthermore, it is possible that amino acids can be replaced by modified amino acids or specific enantiomers. Further modifications are possible of the teaching of WO99/62933 or WO02/38592.

In the state of the art, various possibilities for producing peptides are disclosed. Peptides that are designed proceeding from the inventive peptides, using such methods, are also covered by the inventive teaching. One possibility of generating function-analogous peptides is described, for example, in PNAS USA 1998, Oct. 13; 9521:12179-84, WO 99/62933, and/or WO 02/38592; these teachings are incorporated into the disclosure content of the invention. This means that all peptides, peptide fragments, or structures, which comprise peptides and were generated using the stated methods (proceeding from the inventive peptides) are peptides within the sense of the invention, if they accomplish the inventive task, particularly if they interact with the disease-causing autoantibodies. These autoantibodies can be, for example, agonistic autoantibodies, which activate receptors, or inhibitive antibodies.

In another preferred embodiment of the invention, it is preferred that
the autoantibodies associated with humoral kidney rejection are brought into contact with the peptide comprising a sequence or partial sequence of the second loop of the angiotensin II AT1 receptor.

In another preferred embodiment of the invention, the IgG subclasses are the IgG1 and IgG3 subclasses. Advantageously, specific subclasses are used in order to detect disease-associated autoantibodies in simple and effective manner. In this way, simple and more reliable detection of autoantibodies can be performed, in contrast to the method without the use of specific subclasses. Surprisingly, the subclasses can be assigned to the specific disease profile. Accordingly, essentially no mixture of IgG subclasses is associated with a disease profile, which is surprising in that in the course of the generation of subclasses by Switch and others, biochemical mechanisms of different subclasses are generated.

In another advantageous embodiment, it is preferred that
in the case of humoral kidney rejection, the IgG1 and IgG3 subclasses are used

By means of this embodiment of the invention, an IgG subclass or subclasses can be made available for a specific autoimmune disease or for the autoantibodies, with which autoantibodies can be detected in simple and reliable manner.

In another preferred embodiment of the invention, the autoantibodies are concentrated or purified before being identified, using methods known to a person skilled in the art.

In a special embodiment of the invention, the method for concentrating or purifying the autoantibodies associated with humoral kidney rejection comprises the following steps:
a) Obtaining an IgG fraction from bodily fluid,
b) Bringing the IgG fraction that was obtained into contact with a peptide that comprises a partial sequence of a first or second loop of a G protein-coupled receptor, whereby a mixture is obtained,
c) Incubating the mixture with a carrier that is washed and concentrated, and
d) Eluting the autoantibodies from the concentrated carrier.

Appropriately, a very reliable diagnosis or detection of the humoral kidney rejection autoantibodies becomes possible by the purification or concentration.

In a special embodiment of the invention, the peptide that comprises the sequence or partial sequence of the first and/or second loop comprises:

### AFHYESQ, ENTNIT.

In particular, these peptides can be assigned as follows (see also Table 3):

AT1 humor.
kidney rej. II loop ENTNIT, AFHYESQ

It is, of course, known to a person skilled in the art that, on the basis of the disclosure of specific sequence segments of the loop, sequences that flank the stated sequences in the naturally occurring loop can be used. Furthermore, it is known to a person skilled in the art that the sequences can be modified by deletions, substitutions, additions, insertions, or other biochemical or biophysical processes in such a manner, that individual parameters of these sequences (for example their action as a diagnostic or therapeutic agent) can be improved.

In another preferred embodiment of the invention, the peptide, in particular, is immobilized. In the sense of the invention, immobilization is understood to mean various methods and techniques for fixing the peptides in place on specific carriers. The immobilization can serve, for example, to stabilize the peptides, so that they are not reduced in their activity or disadvantageously modified by biological, chemical, or physical effects, particularly during storage or when mixed as a one-time batch. By means of the immobilization of the peptides, repeated use under technical or clinical routine conditions is possible; furthermore, a sample (preferably blood components) can continuously be mixed with at least one of the inventive peptides. This can be achieved, in particular, by means of various immobilization techniques, whereby binding of the peptides to other peptides or molecules, or to a carrier, takes place in such a manner that the three-dimensional structure, particularly at the center, which mediates the interactions with the autoantibodies, of the corresponding molecules, particularly the peptides, is not changed. Advantageously, the specificity to the autoantibodies of the patients is not lost by means of the immobilization. In the sense of the invention, three fundamental methods for immobilization can be used:

(i) Lateral crosslinking: In lateral crosslinking, the peptides are fixed in place with one another, without any detrimental effect on their activity. They are advantageously no longer soluble because of the lateral crosslinking.

(ii) Binding to a carrier: Binding to a carrier takes place, for example, by means of adsorption, ion binding, or covalent binding. This can also take place within microbial cells or liposomes, or other closed or open structures that contain membranes. The peptides are advantageously not influenced in their activity by being fixed. The peptides can advantageously be used in the clinic, for example, in diagnosis or treatment, multiple times or continuously, in carrier-bound manner.

(iii) Inclusion: Inclusion takes place, in the sense of the invention, in particularly, on a semi-permeable membrane, in the form of gels, fibrils, or fibers. Encapsulated peptides are separated from the surrounding sample solution, by means of a semi-permeable membrane, in such a manner that they advantageously can still react with the autoantibodies or with fragments thereof. Various methods are available for immobilization, such as adsorption on an inert or electrically charged inorganic or organic carrier. Such carriers can be, for example, porous gels, aluminum oxide, betonide, agarose, starch, nylon, or polyacrylamide. In this connection, immobilization takes place by means of physical bonding forces, often with the involvement of hydrophobic interactions and ionic bonds. Such methods are advantageously simple to handle and they influence the conformation of the peptides only to a slight extent. By means of electrostatic binding forces between the charged groups of the peptides and the carrier, the bond can advantageously be improved, for example by means of the use of ion exchangers, particularly Sephadex.

Another method is covalent bonding to carrier materials. For this purpose, the carrier groups can have reactive groups that enter into homeopolar bonds with amino acid side chains. Suitable groups in peptides are carboxy, hydroxy, and sulfide groups, and, in particular, the end-position amino groups of lysines. The surface of microscopic, porous glass particles can be activated by means of treatment with silanes, and subsequently reacted with peptides. Hydroxy groups of natural polymers can be activated with bromine cyan, for example, and subsequently coupled with peptides. Numerous peptides can advantageously enter into direct covalent bonds with polyacrylamide resins. In the inclusion in three-dimensional networks, the peptides are enclosed in ionotrophic gels or other structures known to a person skilled in the art. The pores of the matrix are, in particular, structured in such a manner that the peptides are retained and an interaction with the target molecules is possible. In the case of lateral crosslinking, the peptides are converted into polymer aggregates by means of crosslinking with bifunctional agents. Such structures are gelatinous and easily deformable, and are particularly suitable for use in different reactors. By adding other inactive ingredients, such as gelatin, for example, in crosslinking, the mechanical and bonding properties can advantageously be improved. In microencapsulation, the reaction space of the peptides is restricted, using membranes. Microencapsulation can take place, for example, as border surface polymerization. By means of immobilization during microencapsulation, the peptides become insoluble and therefore can be used again. In the sense of the invention, immobilized peptides are all peptides that are in a state that allows their re-use. The restriction of mobility and solubility of the peptides in chemical, biological, or physical manner advantageously results in low process costs, particularly in the elimination of autoantibodies from blood components.

In another preferred embodiment of the invention, the peptide is bound to a solid phase. Binding of the peptide to the solid phase can take place by way of a spacer. All chemical compounds that have the suitable structural and functional prerequisites for the function of the spacer can be used as spacers, as long as they do not modify the bonding behavior in such a manner that a bond between the autoantibody and the peptide is impaired in disadvantageous manner. In a particularly preferred embodiment of the invention, the peptide comprises amino groups, amides, acetyl groups, biotin groups, markers, spacers, linkers, GKK and/or SGKK. Such structures advantageously allow use of the peptides in apheresis therapy.

In a particularly preferred embodiment of the invention, the linker and/or the spacer comprises α-amino carboxylic acids as well as their homo-oligomers and hetero-oligomers; α,ω-amino carboxylic acids as well as their branched homo-oligomers and hetero-oligomers; other amino acids as well as the linear and branched homo-oligomers and hetero-oligomers; amino-oligoalkoxy alkyl amines; maleinimido carboxylic acid derivatives; oligomers of alkyl amines; 4-alkylphenyl derivatives; 4-oligoalkoxy phenyl or 4-oligoalkoxy phenoxy derivatives; 4-oligoalkyl mercaptophenyl or 4-oligoalkyl mercaptophenoxy derivatives; 4-oligoalkyl aminophenyl or 4-oligoalkyl aminyphenoxy [sic] derivatives; (oligoalkylbenzyl) phenyl or 4-oligoalkylbenzyl phenoxy derivatives as well as 4-oligoalkoxy benzyl phenyl or 4-oligoalkoxybenzyl phenoxy derivatives; trityl derivatives; benzyloxyaryl or benzyloxyalkyl derivatives; xanthen-3-yl oxyalkyl derivatives; (4-alkyl phenyl) or ω-(4-alkyl phenoxy) alkanic acid derivatives; oligoalkyl phenoxy alkyl or oligoalkoxy phenoxy alkyl derivatives; carbamate derivatives; amines; trialkyl silyl or dialkyl alkoxy silyl derivatives; alkyl or aryl derivatives and/or combinations thereof.

In another particularly preferred embodiment of the invention, the immobilized peptides are modified by means of deletion, addition, substitution, translocation, inversion and/or insertion.

The invention also relates to a peptide selected from the group ENTNIT and/or AFHYESQ. Use as a therapeutic active ingredient in the sense of the invention means the use of the peptide or peptides in the entire field of medicine, preferably for the diagnosis and treatment of the autoimmune disease humoral kidney rejection.

It is known to a person skilled in the art that he/she can generate additional function-analogous peptides on the basis of the disclosed peptides, as diagnosis and/or therapy agents. These function-analogous peptides are included in the inventive teaching. In particular, reference is made to the dissertation PNAS USA 1998, Oct. 13; 9521:12179-84, WO 00/6293 [sic] and/or WO 02/38592, which are incorporated into the disclosure content of the inventive teaching.

In a preferred embodiment of the invention, the peptide is bound by autoantibodies of patients having humoral kidney rejection. A person skilled in the art can make diagnosis and treatment methods available from this disclosure, by means of routine experiments.

The invention also relates to recognition molecules that are directed at the inventive peptide. Preferably, the recognition molecules are antibodies, antisense constructs and/or a chelators. The recognition inventive molecules can be antibodies that are directed against autoantibodies that particularly induce humoral kidney rejection.

The invention also relates to a pharmaceutical composition that comprises the peptides and/or the recognition molecules, if applicable with a pharmaceutically compatible carrier. The pharmaceutical composition can be used, in particular, as a medication. For this purpose, it is possible, for example, to modify the peptides, by means of cyclization or other methods known to a person skilled in the art, in such a manner that they cannot be destroyed by peptide-decomposing structures inherent to the body, such as serum proteases, for example. By means of the use of the peptides or recognition inventive molecules, it is possible to neutralize the autoantibodies in vivo or ex vivo, or in vitro. In vitro neutralization is advantageous, for example, in the investigation of autoimmune diseases in tissue cultures or cell cultures. In the case in vivo neutralization, the medications are administered to the patient directly, while in the case of ex vivo neutralization, the blood is passed out of the body by way of a loop (for example in the form of a hose circulation), consequently brought into contact with the medication, and after neutralization has taken place, the autoantibody is passed back into the organism, particularly the human patient. In the sense of the invention, both those pharmaceutical compositions that are used for therapeutic and prophylactic purposes and those pharmaceutical compositions that can be used as diagnostic agents are considered to be medications.

Medications or pharmaceutical compositions, terms that are used synonymously here, are, pursuant to the invention, substances and formulations of substances that are intended to heal, alleviate, or prevent diseases, illnesses, bodily harm, or pathological symptoms, by being used on or in the human body. Medicinal ancillary substances are, pursuant to the invention, those substances that are used for the production as active ingredients of medications. Pharmaceutical technology ancillary substances serve for suitable formulation of the medication or the pharmaceutical composition, and can even be removed later, if they are only required during the production process, or can be part of the pharmaceutical composition as a pharmaceutically compatible carrier. The formulation of the medication or formulation of the pharmaceutical composition takes place, if necessary, in combination with the pharmaceutically compatible carrier and/or dilutant. Examples of suitable pharmaceutically compatible carriers are known to a person skilled in the art and comprise, for example, phosphate-buffered saline solutions, water, emulsions such as oil/water emulsions, for example, various types of detergents, sterile solutions, etc. Medications that comprise such carriers can be formulated by means of known, conventional methods. These medications or pharmaceutical compositions can be administered to an individual in a suitable dose, for example in a range from 1 μg to 10 g peptides per day and patient. In this connection, doses of 1 mg to 1 g are preferred. Administration of the fewest and lowest possible doses is preferred, and furthermore, a one-time dose is preferred. Administration can take place in different ways, for example in intravenous, intraperitoneal, intrarectal, intragastrointestinal, intranodal, intramuscular, local, but also subcutaneous, intradermal manner, or on the skin or by way of the mucous membranes. Administration of nucleic acids that code for the inventive peptide can also take place in the form of gene therapies, for example by way of viral vectors. The type of dosage and the administration path can be determined by the treating physician, in accordance with the clinical factors. It is known to a person skilled in the art that the type of dosage is dependent on various factors, such as, for example, height, body surface, age, gender, or the general health of the patient, but also on the special agent that is being administered, the duration and method of administration, and on other medications that might be administered in parallel. A person skilled in the art can orient himself/herself on the basis of the usual standard values as well as special teachings, for example the teaching of EP 1 085 955, which is incorporated into the disclosure content of the invention. Furthermore, it is known to a person skilled in the art that he/she can first diagnose the concentration of the autoantibodies with the inventive peptides, in order to determine the necessary concentration of the medication.

The pharmaceutical compositions or the medication particularly comprise a pharmacological substance that contains one or more inventive peptides or recognition molecules and/or nucleic acid molecules that code for them, in a suitable solution or administration form. These can be administered either alone, with the corresponding ancillary substances described under medications or pharmaceutical compositions, or in combination with one or more adjuvants, for example QS-21, GPI-0100 or other saponines, water/oil emulsions such as montanide, for example, adjuvants, polylysine, polyarginine compounds, DNA compounds such as CpG, for example, detox, bacterial vaccines such as typhus vaccine or BCG vaccine, for example, salts such as calcium phosphates, for example, and/or another suitable substance for enhancing effect; preferably immune-stimulating molecules such as interleukins, for example IL-2, IL-12, IL-4 and/or growth factors, for example GM-CSF. These are mixed with the inventive peptides or recognition molecules, using known methods, and administered in a suitable formulation and dosage. Formulations, dosages, and suitable components are known to a person skilled in the art.

The pharmaceutical composition or the medication can, of course, also be a combination of two or more of the inventive pharmaceutical compositions or medications, as well as a combination with other medications, such as, for example, antibody therapies, chemotherapies, or radiotherapies, which are administered or applied at the same time or separately, in suitable manner. The production of the medications or pharmaceutical compositions takes place using known methods.

The invention also relates to a kit that comprises the inventive peptide, the inventive recognition molecules, and/or the inventive pharmaceutical composition, if necessary with instructions for combining the contents of the kit and/or for making available a formulation for a recipient and an algorithm for administration of the formulation, in other words in what dose or at what time intervals individual components of the kit are to be administered to a patient. The recipient in the sense of the invention can, however, also be a cell or a tissue in vivo, ex vivo, or in vitro. The information can be, for example, a package insert, but also information that can be called up by the use by phone or via the Internet. The algorithm for administration of the formulation particularly includes instructions regarding the diagnostic and/or therapeutic method for treatment of a patient. This can be a single-stage or also multi-stage method, as well as methods that are carried out in the absence or the presence of the physician. This means that the therapy plan, i.e. the information about the latter, is preferably a component of the kit.

The invention also relates to a device for chromatography that comprises the inventive peptides.

In a preferred embodiment, the peptides are bound to a solid phase, for example, within the chromatography system.

The inventive device can be used, in particular, to eliminate the autoantibodies from fluids of a patient, i.e. to neutralize the autoantibodies. This method is known to a person skilled in the art under the term of immune adsorption or apheresis therapy. Using immune adsorption, immunoglobulins are removed from the patient's blood. Advantageously, this immune adsorption treatment can be performed on an in-patient or out-patient basis. It can be provided that the device, particularly the so-called adsorber, is a component of an extracorporal blood circulation. In this connection, blood is continuously or discontinuously taken from a larger blood vessel of the patient, particularly an arm vein, and separated into individual components, such as the cellular and humoral components, for example, by means of filtration or centrifugation. A significant component of the blood that is obtained thereby is, in particular, blood plasma. The blood plasma can advantageously be passed through the inventive device and, after adsorption of the autoantibodies, be given back to the patient, together with the blood components separated previously, particularly the cellular components, particularly through a different arm or leg vein. It can furthermore be provided that the peptides are immobilized on a sepharose matrix. This matrix can be placed into a container that has a volume from 10 to 400 ml. The blood plasma of the patient can then be passed over this matrix, whereby the autoantibodies are bound and can therefore be eliminated from the blood plasma. A person skilled in the art is aware of various possibilities for making available such peptides fixed on a solid phase, for example in the form of (i) adsorption columns capable of regeneration, in the form of (ii) double columns, as well as in the form of (iii) columns for one-time use. The various rinsing and elution solutions that allow a high level of efficiency of the treatment can easily be determined by a person skilled in the art, by means of routine experiments. By means of making available the inventive teaching, particularly the inventive peptides, various possibilities are disclosed to a person skilled in the art, for using these in vivo, ex vivo, and in vitro, for the prophylaxis, diagnosis, therapy, as well as follow-up treatment of humoral kidney rejection. Further embodiments are known to a person skilled in the art from WO 02/38592, EP 1 214 350, and WO 99/56126, which are incorporated into the disclosure content of the inventive teaching.

The invention also relates to the use of the inventive peptides, the inventive pharmaceutical composition, the inventive kit, and/or the inventive device, for the prophylaxis, diagnosis, therapy, monitoring of progression and/or follow-up treatment of humoral kidney rejection.

The invention also relates to the use of the inventive peptides, the inventive pharmaceutical composition, the inventive kit, and/or the inventive device, for the production of a medication for the treatment of humoral kidney rejection.

The invention also relates to the use of the inventive peptides, the inventive pharmaceutical composition, the inventive kit, and/or the inventive device, for screening medications. Screening of medications can comprise, for example, the identification of substances, particularly peptides, proteins, carbohydrates and/or lipids, which interact with the peptides. An interaction can be, for example, binding to these peptides, but also activation or inhibition of or by the stated peptides. Accordingly, a medication could be a structure, for example, that binds to the peptides in the body of a patient, and therefore to the corresponding loops, and thus competes for a binding site with the autoantibodies that occur there. By means of the disclosure of the inventive teaching, particularly by way of the disclosure of the connection between a disease and the binding location of the autoantibodies, a person skilled in the art can screen different medications. Screening of medications on the basis of disclosed targets belongs to the general knowledge of a person skilled in the art, and takes place by means of routine experiments; reference is made to the corresponding standard works in molecular biology and pharmacology.

The invention also relates to a method for treating humoral kidney rejection, by means of binding and/or removing autoantibodies by means of inventive peptides that are bound to a solid phase. By means of the peptides bound to the solid phase, the autoantibodies are bound, complexed and/or neutralized on the solid phase.

In a special embodiment of the treatment method, it is preferred that the autoantibodies are directed against angiotensin II AT1 receptors in the case of humoral kidney rejection.

In the following, the invention will be explained in greater detail using an example, without being restricted to this example.

### EXAMPLE

### Identification of Angiotensin II AT1 Receptor Autoantibodies

Spontaneously beating, cultivated cardiomyocytes of newborn rats are a very useful model for studying the effect of autoantibodies.

Wallukat et al., 2001, already reported about studies of β₁ adrenoreceptor autoantibodies. This report concerns itself with angiotensin II AT₁ receptor autoantibodies in preeclamptic women. Preeclampsia is an illness that makes itself known by an increase in blood pressure and can result in death of the mother and the fetus. Dechend et al., 2000, were able to demonstrate the manifestation of agonistic antibodies against angiotensin AT₁ receptors that frequently occur in preeclamptic women. It was possible to explain many of the pathophysiological characteristics of preeclampsia with the activation of the AT₁ receptor by agonistic autoantibodies. The findings of Wallukat et al., 1999, show that immunoglobulin fractions and affinity-purified antibodies of preeclamptic women can stimulate the AT₁ receptor of cultivated cardiomyocytes. The beats per minute are reduced by adding Losartan (1 μM). It was possible to show, by means of neutralization experiments, that the IgG subclass 3 is responsible for the increase in heart rate.

Of these findings, an enzyme-coupled immune test for identifying angiotensin II AT₁ receptor autoantibodies (anti-AT₁-AAB) was developed.

First: Peptide solutions corresponding to the amino acid sequence of the second loop of the human AT₁ receptor (Sm 1986/1, 100 μg/ml), were incubated with anti-AT₁-AAB (1:1; vol./vol.) for 1 hour at 4° C. Anti-AT₁-AABs were produced by means of ammonium sulfate precipitation from waste fluids during birth (blood and isotonic saline solution). These samples had a stronger concentration than pure serum samples.

Second: This mixture was incubated with washed streptavidin-coated magnetic particles for 1 hour at 4° C.

Third: To separate the IgG/peptide mixture, the magnetic particles were washed three times with washing buffer (20 mM potassium phosphate buffer, 0.15 M CnCl, pH 7.5). The separation or washing can easily be performed using a magnet concentration apparatus (Dynal). Non-specific binding sites were blocked with 1% bovine serum albumin in washing buffer.

Fourth: The magnetic particles were incubated with a solution of horseradish peroxidase-marked antibodies against human IgG3 (1:200, 1 hour, room temperature).

Fifth: The particles were treated in the dark, at room temperature, for 30 min, using a standardized, ready-to-use solution of TMB (tetramethyl benzidine). The color reaction (blue-green) was stopped using 0.1 N HCl (yellow-orange). The optical density values were measured in a microplate reader (Anthos HTII) at 492 nm (reference filter 620 nm). The results are listed in Table 1.

The same peptide of the human AT₁ receptor (Sm 1986/1) was used to purify anti-AT₁-AABs. IgG solutions were mixed with peptide solution (100 μg/ml, 1:1; vol./vol.) and incubated at 4° C. for one hour. The streptavidin-coated magnetic particles that had been washed three times were added (300 μl). The particles were collected using a magnet concentration apparatus. The top fractions were carefully removed and stored in ice. The magnetic particles were washed three times and eluted with 3 M potassium thiocyanate solution for 15 min at room temperature. After magnetic concentration, the solutions were carefully separated and dialyzed in phosphate-buffered solution, against NaCl (0.9%), together with the first top fraction. After replacement five times within three days, the protein content was determined on the basis of the optical density (280 nm). The chronotropic effect of the top fraction and eluate on primary cultivated cardiomyocytes of newborn rats (bioassay) was recorded using an imaging computer system (IMAGOQANT).

Table 2 shows the reproducibility of the purification method. Six of the six purified anti-AT₁-ABBs showed the increase in heart rate/min (>24.4). Cultures treated with top fraction, on the other hand, result in no change or only moderate changes in the heart rate (<10.0).

The method of coimmunoprecipitation of the AT₁ receptor was similar to the method for the β₁ adrenoreceptor (Wallukat, 2001). The differences are: lysed membranes of transfected CHO cells (Couchon, 1997) were used for the coimmunoprecipitation. The lysed membranes should be freshly produced. The proteins were identified using an antibody against a peptide having the sequence of the N-terminal part of the AT₁ receptor, which had been produced in rabbits (N10, 1:100, Santa Cruz), and identified by means of Western blot and ECL system, with anti-rabbit IgG peroxidase conjugates (1:10,000, Sigma).

FIG. 1 shows the results of the Western blot. It was possible to precisely detect a band (molecular weight >40.0 kDa), using internal positive samples (lysed membranes of transfected CHO cells and human placenta tissue). In earlier experiments (Neichel, non-published data), it was possible to block this band by means of the peptides that were used for producing the N10 antibodies. This band was missing in pure sepharose samples and in the top fractions of the purification experiments.

The results show the usefulness of the imaging computer system IMAGOQANT in detecting the increase in the beats/min caused by AT₁-ABBs in patients having preeclampsia. The enzyme-coupled immune test should also be checked with sera from preeclampsia patients and healthy donors. The purified AT₁-ABBs can be used for further investigation of the pathogenesis of preeclampsia.

TABLE 1
Measurement of the AT1 autoantibodies using an enzyme-coupled immune
test

| | | Optical density |
|---|---|---|
| | | (OD, 492 nm) |
| IgG | n | Range |
| Healthy test subjects (controls) | 3 | 0.036-0.069 |
| Preeclamptic woman | | |
| Positive | 15 | 0.071-0.786 |
| Negative | 4 | 0.021-0.069 |

TABLE 2
Influence of top fractions and eluates of the magnetic particles on the
heart rate of cultivated cardiomyocytes of newborn rats

| Patient/ | | | | | | |
|---|---|---|---|---|---|---|
| Date of experiment | Samples | OD | μg/ml | Bioassay (increase in beats/min) | | |
| D. | top fraction | 4.300 | 3071.4 | 6.0 ± 0.0 | 6.0 ± 0.0 | 10.0 ± 1.6 |
| Mar. 19, 2002 | | | | | | |
| | eluate | 0.086 | 61.4 | 12.8 ± 1.6 | 27.6 ± 2.0 | 34.4 ± 1.2 |
| D. | top fraction | 6.820 | 4871.4 | −1.6 ± 0.8 | 4.0 ± 1.2 | 6.4 ± 1.2 |
| May 27, 2002 | | | | | | |
| | eluate | 0.033 | 23.6 | 12.1 ± 2.4 | 18.9 ± 0.8 | 24.5 ± 0.8 |
| D. | top fraction | | | 3.3 ± 0.8 | 3.2 ± 0.8 | 4.7 ± 1.6 |
| Jun. 3, 2002 | | | | | | |
| | eluate | 0.0104 | 74.3 | 11.1 ± 1.2 | 15.2 ± 2.0 | 33.9 ± 2.0 |

± SD from average value

TABLE 3
Autoantibodies against G protein-coupled receptors
Information concerning the epitopes and IgG subclass

| Antibodies | | | |
|---|---|---|---|
| against rec. | Disease | Epitope | IgG subclass |
| Ang. II AT1 | Humoral kidney rejection | 2^{nd} loop | IgG1 and IgG3 |

### Key

FIG. 1: Western blot of the coimmunoprecipitation of the angiotensin AT₁ receptor Track 1 protein A/sepharose; 2 preeclamptic patient D. without purification; 3 KSCN eluate; 4 top fraction; 5 lysed CHO membrane; 6 lysed placenta tissue.

1-35. (canceled)

36. Method for detecting autoantibodies associated with humoral kidney rejection, comprising the following steps:
a) Bringing bodily fluid into contact with a denaturing agent,
b) Bringing the precipitated fraction into contact with a peptide, particularly one comprising biotin, which comprises a partial sequence of the first and/or second loop of the receptor, whereby a mixture is formed,
c) Incubating the mixture with a carrier coated with avidin or streptavidin,
d) Washing the materials of the carrier,
e) Incubating the carrier with anti-IgG antibody subclasses, whereby the anti-IgG antibody is marked, and
f) Carrying out an enzyme reaction or color reaction.

37. Method of claim 36,
characterized in that
the autoantibodies are directed against an angiotensin II AT1 receptor.

38. Method of claim 36,
characterized in that—the autoantibodies associated with humoral kidney rejection are brought into contact with the peptide comprising a sequence or partial sequence of the second loop of the angiotensin II AT1 receptor

39. Method of claim 36,
characterized in that
the IgG subclasses are IgG1 and IgG3 subclasses.

40. Method of claim 36,
characterized in that
the autoantibodies are concentrated or purified before being identified.

41. Method of claim 36,
characterized in that
the method for concentrating or purifying the autoantibodies comprises the following steps:
a) Obtaining an IgG fraction from bodily fluid,
b) Bringing the IgG fraction that was obtained into contact with a peptide that comprises a partial sequence of a first or second loop of a G protein-coupled receptor, whereby a mixture is obtained,
c) Incubating the mixture with a carrier that is washed and concentrated, and
d) Eluting the autoantibodies from the concentrated carrier.

42. Method of claim 36,
characterized in that
the peptide that comprises the sequence or partial sequence of the first and/or second loop is selected from the group consisting of AFHYESQ and ENTNIT.

43. Method of claim 36,
characterized in that
the peptide comprises amino groups, amides, acetyl groups, biotin groups, markers, spacers, linkers, GKK and/or SGKK.

44. Peptide selected from the group consisting of AFHYESQ and ENTNIT, for use as a medicinal active substance in the treatment of humoral kidney rejection.

45. Peptide of claim 44,
characterized in that
the peptide is bound by autoantibodies of patients having humoral kidney rejection.

46. Pharmaceutical composition for use in the treatment of humoral kidney rejection, comprising a peptide selected from the group consisting of AFHYESQ and ENTNIT and/or a recognition molecule directed against the peptide.

47. Kit comprising a peptide for use in the treatment of humoral kidney rejection selected from the group consisting of AFHYESQ and ENTNIT, a recognition molecule directed against the peptide, and/or a pharmaceutical composition comprising the peptide and/or the recognition molecule.

48. Chromatography device comprising peptides associated with humoral kidney rejection selected from the group consisting of AFHYESQ and ENTNIT, and/or recognition molecules directed against the peptide.

49. Method for treating humoral kidney rejection by means of binding and/or removing antibodies by means of peptidesselected from the group consisting of AFHYESQ and ENTNIT, bound to a solid phase.

50. Method of claim 49,
characterized in that
the autoantibodies are directed against angiotensin II AT1 receptors.

51. Method for the prophylaxis, diagnosis, therapy, monitoring the progression as well as follow-up treatment of humoral kidney rejection, comprising the step of using one or more chosen from the following: (a) Peptide selected from the group consisting of AFHYESQ and ENTNIT (b) a recognition molecule directed against said peptide (c) a pharmaceutical composition comprising said peptide and said recognition molecule (d) a kit comprising one of said peptide, said recognition molecule or said pharmaceutical composition, optionally with instructions for combining the contents of the kit and/or for making available a formulation and (e) a chromatography device comprising said peptide or said recognition molecule.

52. Method for the production of a medication for the treatment of humoral kidney rejection, comprising the step of using one or more chosen from the following (a) Peptide selected from the group consisting of AFHYESQ and ENTNIT (b) a recognition molecule directed against said peptide (c) a pharmaceutical composition comprising said peptide and said recognition molecule (d) a kit comprising one of said peptide, said recognition molecule or said pharmaceutical composition, optionally with instructions for combining the contents of the kit and/or for making available a formulation and (e) a chromatography device comprising said peptide or said recognition molecule.

53. Method for screening medications for the treatment of humoral kidney rejection, comprising the step of one or more chosen from the following (a) Peptide selected from the group consisting of AFHYESQ and ENTNIT (b) a recognition molecule directed against said peptide (c) a pharmaceutical composition comprising said peptide and said recognition molecule (d) a kit comprising one of said peptide, said recognition molecule or said pharmaceutical composition, optionally with instructions for combining the contents of the kit and/or for making available a formulation and (e) a chromatography device comprising said peptide or said recognition molecule.

54. Method for detecting, binding, complexing or neutralizing of autoantibodies associated with humoral kidney rejection, directed against angiotensin II AT1 receptor, comprising the step of using a peptide selected from the group consisting of AFHYESQ and ENTNIT.


SEQUENCE LISTING (16 sequences)

SEQ ID NO: 1 - protein, 6 aa, Artificial
  Description of the artificial sequence peptide
EYGSFF

SEQ ID NO: 2 - protein, 6 aa, Artificial
  Description of the artificial sequence peptide
SFFCEL

SEQ ID NO: 3 - protein, 7 aa, Artificial
  Description of the artificial sequence peptide
ARRCYND

SEQ ID NO: 4 - protein, 6 aa, Artificial
  Description of the artificial sequence peptide
PKCCDF

SEQ ID NO: 5 - protein, 5 aa, Artificial
  Description of the artificial sequence peptide
AESDE

SEQ ID NO: 6 - protein, 6 aa, Artificial
  Description of the artificial sequence peptide
CYIQFF

SEQ ID NO: 7 - protein, 6 aa, Artificial
  Description of the artificial sequence peptide
EDGECY

SEQ ID NO: 8 - protein, 25 aa, Artificial
  Description of the artificial sequence peptide
VRTVEDGECYIQFFSNAAVTFGTAI

SEQ ID NO: 9 - protein, 7 aa, Artificial
  Description of the artificial sequence peptide
AFHYESQ

SEQ ID NO: 10 - protein, 6 aa, Artificial
  Description of the artificial sequence peptide
ENTNIT

SEQ ID NO: 11 - protein, 6 aa, Artificial
  Description of the artificial sequence peptide
FWAFGR

SEQ ID NO: 12 - protein, 7 aa, Artificial
  Description of the artificial sequence peptide
GRAFCDV

SEQ ID NO: 13 - protein, 7 aa, Artificial
  Description of the artificial sequence peptide
ITEEAGY

SEQ ID NO: 14 - protein, 6 aa, Artificial
  Description of the artificial sequence peptide
ERFCGI

SEQ ID NO: 15 - protein, 6 aa, Artificial
  Description of the artificial sequence peptide
GRIFCD

SEQ ID NO: 16 - protein, 8 aa, Artificial
  Description of the artificial sequence peptide
ITTCHDVL